(19) United States
(12) Patent Application Publication
(10) Pub. No.: US 2020/0289476 A1
(43) Pub. Date: Sep. 17, 2020

(54) LIQUID FORMULATIONS OF RILUZOLE FOR ORAL AND INTRAVENOUS USE

(71) Applicants: University of Houston System, Houston, TX (US); The Methodist Hospital Research Institute, Houston, TX (US)

(72) Inventors: Diana S-L. Chow, Houston, TX (US); Mahua Sarkar, Houston, TX (US); Robert G. Grossman, Houston, TX (US)

(73) Assignees: University of Houston System, Houston, TX (US); The Methodist Hospital Research Institute, Houston, TX (US)

(21) Appl. No.: 16/767,706

(22) PCT Filed: Nov. 28, 2018
(86) PCT No.: PCT/US2018/062755

§ 371 Date: May 28, 2020

Related U.S. Application Data
(60) Provisional application No. 62/591,539, filed on Nov. 28, 2017.

(51) Int. Cl. A61K 31/428; A61K 47/34; A61K 47/10; A61K 9/00

(52) U.S. Cl. CPC A61K 31/428; A61K 9/0053; A61K 47/10; A61K 47/34

(57) ABSTRACT

Embodiments of the present disclosure pertain to compositions that include a liquid formulation comprising a molecule selected from the group consisting of riluzole, a derivative of riluzole, an analog of riluzole, a pharmaceutical equivalent of riluzole, a benzothiazole-based molecule, combinations thereof, and salts thereof. The liquid formulations may also include a solubilizing agent. Additional embodiments of the present disclosure pertain to methods of treating a condition or disease in a subject by administering to the subject a composition of the present disclosure. The condition or disease to be treated may include spinal cord injury, and the administration may occur by parenteral administration. In some embodiments, the administered molecule may have an absorption half-life of less than 1.5 hours, an elimination half-life of more than 10 hours, and a bioavailability of more than 65%. Additional embodiments of the present disclosure pertain to methods of making the compositions.

### CROSS-REFERENCE TO RELATED APPLICATIONS

This application claims priority to U.S. Provisional Patent Application No. 62/591,539 filed on Nov. 28, 2017. The entirety of the aforementioned application is incorporated herein by reference.

### FIELD OF THE INVENTION

The present disclosure relates generally to the field of medicine and medical treatment.

### BACKGROUND OF THE DISCLOSURE

Riluzole (6-trifluoromethoxy-2-benzothiazolamine) is a benzothiazole class of drug and is approved by the FDA for the treatment of Amyotrophic Lateral Sclerosis (ALS), a progressive motor neurodegenerative disorder. The positive and improved survival of ALS patients can be attributed to the anti-glutamatergic activity of riluzole.

Riluzole is a voltage sensitive sodium channel blocker. It prevents the intracellular influx of sodium and calcium ions, thereby preventing the calcium dependent neuronal release of excitotoxic glutamate. It also prevents the presynaptic release of glutamate and increases glutamate uptake by activating glutamate transporters. The overall anti-glutamatergic and anti-excitotoxic activity makes it a suitable candidate for many neurological conditions. One such devastating neurological condition is acute spinal cord injury (SCI), and riluzole is currently being investigated for the treatment of SCI.

A phase I trial of riluzole in spinal cord injured patients has been successfully completed. Currently, a double-blinded placebo-controlled Phase II/III clinical trial is underway to evaluate riluzole's efficacy for the treatment of SCI.

Riluzole is commercially available as a capsule-shaped, white, film-coated tablet containing 50 mg of riluzole. The recommended daily dose is 100 mg (50 mg taken orally twice daily). The same dose (i.e., 50 mg twice daily) and oral route of administration have been used in spinal cord injured patients during Phase I and Phase II/III, except that a loading dose of 100 mg twice daily was administered for the first 24 hours in the Phase II/III trial.

However, a large number of SCI patients suffer from dysphagia (swallowing abnormalities) caused by severe motor dysfunction following a spinal cord injury. Thus, there remains a need for a liquid formulation of riluzole, which may be more suitable for patients with swallowing difficulties.

### SUMMARY OF THE DISCLOSURE

In some embodiments, the present disclosure pertains to a composition. The composition includes a liquid formulation comprising a molecule selected from the group consisting of riluzole, a derivative of riluzole, an analog of riluzole, a pharmaceutical equivalent of riluzole, a benzothiazole-based molecule, combinations thereof, and salts thereof.

In some embodiments, the molecule has a concentration of more than 5 mg/ml. In some embodiments, the molecule has a concentration of at least about 10 mg/ml. In some embodiments, the molecule includes riluzole. In some embodiments, the molecule is dissolved in the liquid formulation.

In some embodiments, the liquid formulation also includes a solubilizing agent. In some embodiments, the solubilizing agent includes, without limitation, polyethylene glycol, glycerin, propylene glycol, ethanol, sorbitol, polyoxyethylated glycerides, polyoxyethylated oleic glycerides, polysorbates, sorbitan monooleate, hydroxypropyl-beta-cyclodextrin, polyoxyl 40 hydrogenated castor oil, polyoxyl hydroxystearates, and combinations thereof.

In some embodiments, the liquid formulation has sufficient homogeneity for parenteral administration. In some embodiments, the liquid formulation is a colorless solution at room temperature with no visible particles.

In some embodiments, the molecule in the liquid formulation has a t₉₀ room temperature stability of more than 10 months. In some embodiments, the molecule in the liquid formulation has a t₉₀ room temperature stability of more than 15 months.

Additional embodiments of the present disclosure pertain to methods of treating a condition or disease in a subject by administering to the subject a composition of the present disclosure. In some embodiments, the condition or disease to be treated includes, without limitation, spinal cord injury (SCI), swallowing abnormalities, dysphagia, neurological disease or condition, amyotrophic Lateral Sclerosis (ALS), and combinations thereof.

In some embodiments, the administration of the composition to the subject occurs by parenteral administration. In some embodiments, the administered molecule has an absorption half-life of less than 1.5 hours or less than 1 hour. In some embodiments, the administered molecule has an elimination half-life of more than 10 hours or more than 15 hours. In some embodiments, the administered molecule has a bioavailability of more than 65% or more than 80%.

In some embodiments, the administered molecules of the present disclosure can become localized in the central nervous system. For instance, in some embodiments, the brain (μg/g) to plasma (μg/ml) ratio of the administered molecule is more than 3.5 after 24 hours of administration. In some embodiments, the spinal cord (μg/g) to plasma (μg/ml) ratio of the molecule is more than 6.5 after 24 hours of administration.

Additional embodiments of the present disclosure pertain to methods of making the compositions of the present disclosure by associating a molecule of the present disclosure with a liquid formulation of the present disclosure. In some embodiments, the association occurs by a method that includes, without limitation, mixing, stirring, heating, milling, compressing, and combinations thereof. In some embodiments, the methods of the present disclosure also include a step of encapsulating the formed compositions in a carrier, such as a capsule.

### DESCRIPTION OF THE DRAWINGS

FIG. 1A illustrates a method of administering a composition of the present disclosure to a subject.

FIG. 1B illustrates a method of making the compositions of the present disclosure.

FIG. 2 depicts a perturbation plot showing the effect of concentrations of PEG 400(A), Propylene Glycol (B) and Glycerin (C) on amount of riluzole solubilized in 5 mL (Y).

FIGS. 3A and 3B depict a response surface plot of the effect of concentrations of PEG 400 and Propylene Glycol (PG) on amount of riluzole dissolved (FIG. 3A) and a desirability profile for the selected optimum composition (FIG. 3B).

FIGS. 4A and 4B depict a degradation profile of riluzole from a co-solvent formulation at different temperatures (linear scale) (FIG. 4A), and an Arrhenius plot for predicting t₉₀ of riluzole from a co-solvent formulation (FIG. 4B).

FIG. 5 depicts a hemolytic potential of an optimum co-solvent formulation containing riluzole (solid line) and a blank co-solvent formulation without riluzole (dotted line).

FIG. 6 depicts plasma concentration (normalized by dose)-time profiles of riluzole after the following modes of administration in rats: single oral (10 mg/kg) administrations of co-solvent formulations; administration of tablets (crushed and suspended); and intravenous (5 mg/kg) administration of co-solvent formulations (n=5 for each group). Each data point is expressed as Mean±SD. The inset graph shows the oral plasma profiles of riluzole from the co-solvent formulation and crushed tablet groups.

FIG. 7 shows the pharmacokinetic profiles of riluzole in the plasma, brain and spinal cord of rats (0.5, 3, 6, 9 and 24 hr) after a single dose administration of a co-solvent formulation of riluzole (10 mg/kg, oral) (N=3 at each time point). Each data point is expressed as Mean±SD.

### DETAILED DESCRIPTION

All references, publications, and patents cited herein are incorporated by reference in their entirety as though they are fully set forth. Unless defined otherwise, technical and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application. One skilled in the art will recognize many methods and materials similar or equivalent to those described herein, which could be used in the practice of the present invention. The present invention is in no way limited to the methods and materials described.

Riluzole is a benzothiazole class of drug. Riluzole's anti-glutamatergic and anti-excitotoxic activity makes it a suitable candidate for many neurological conditions. One such devastating neurological condition is acute spinal cord injury (SCI), and riluzole is currently being investigated for the treatment of SCI. A phase I trial of riluzole (Clinical Trial No. NCT 00876889) in spinal cord injured patients has been successfully completed. Currently, a double-blinded placebo-controlled Phase II/III clinical trial (Clinical trial Reg. No. NCT01597518) is underway to evaluate Riluzole's efficacy for the treatment of SCI. In addition, riluzole is approved for the treatment of Amyotrophic Lateral sclerosis (ALS).

Riluzole is commercially available as a capsule-shaped, white, film-coated tablet containing 50 mg of riluzole. The recommended daily dose is 100 mg (50 mg taken orally twice daily).

A large number of SCI patients suffer from dysphagia (swallowing abnormalities) caused by severe motor dysfunction following a spinal cord injury. The extent of dysphagia varies amongst this patient population, and can range from mild to extreme difficulty in swallowing. This complicates the administration of solid oral dosage formulations like tablets and capsules. In such cases, manipulations and alterations of formulations, such as crushing tablets or opening of capsules, are the common practice to facilitate the swallowing. These manipulations can have severe consequences, such as erratic bioavailability and reduced efficacy, due to incomplete or inaccurate dose of the medication.

During the Phase I study of riluzole for the treatment of SCI, acute SCI patients received 50 mg riluzole either orally or enterally through a nasogastric tube every 12 hours. When patients were unable to take the tablet orally, the tablet was crushed and dispersed in water and administered through a nasogastric tube. Crushing a tablet might cause sub therapeutic dosing, since the complete dose is not delivered.

Powder losses during crushing and inefficient extraction of a dispersion are the possible reasons for drug loss. Moreover, dosing with crushed tablets is reported to be associated with erratic pharmacokinetics, altered rate of absorption and reduced bioavailability. This indicates the need for an alternative dosage form of riluzole for a given target population of spinal cord injured patients.

Moreover, SCI patients are critical care patients and enteral administration of medications is difficult. Lack of an alternative formulation, such as parenteral formulation, restricts the usage of required medication. Since riluzole is given to critical care SCI patients, a parenteral formulation will be desirable.

In particular, a liquid formulation would be a rational choice for critically ill SCI patients. Additionally, a liquid preparation offers the advantages of homogeneity, dose consistency, flexibility of dose adjustment, and enhanced bioavailability of poorly soluble drugs.

Recently, a liquid oral suspension of riluzole (Teglutik®, 5 mg/ml) was launched in the United Kingdom by Martindale Pharma Limited. The suspension offers advantages of a readily available preparation for administration where no manipulation, premixing or dilution is required. However, the Teglutik® suspension dosage form has its own challenges. For instance, the homogeneity of the suspension has to be ensured every time prior to administration by manually shaking it and by visual inspection. In addition, there is no parenteral formulation of riluzole that is available to date. Therefore, use of riluzole in many critical care patients, such as SCI patients, is inadequate.

As such, a need exists for the development of improved liquid formulations of riluzole that have sufficient homogeneity for numerous routes of administrations, such as parenteral administration. A need also exists for the development of improved liquid formulations of riluzole that can contain high dosages of riluzole. Various embodiments of the present disclosure address the aforementioned needs.

In some embodiments, the present disclosure pertains to compositions with a liquid formulation, where the liquid formulation includes a molecule. In some embodiments, the molecule in the liquid formulation includes, without limitation, riluzole, a derivative of riluzole, an analog of riluzole, a pharmaceutical equivalent of riluzole, a benzothiazole-based molecule, combinations thereof, and salts thereof. Additional embodiments of the present disclosure pertain to kits that include the compositions of the present disclosure.

The compositions of the present disclosure may be in various forms. For instance, in some embodiments, the compositions of the present disclosure may be encapsulated, tableted or prepared in an emulsion or syrup.

Further embodiments of the present disclosure pertain to methods of treating a condition or disease in a subject by administering to the subject a composition of the present disclosure. Additional embodiments of the present disclosure pertain to methods of making the compositions of the present disclosure.

As set forth in more detail herein, the present disclosure can have numerous embodiments. In particular, the compositions of the present disclosure can include various types of liquid formulations that contain various molecules. Moreover, various methods may be utilized to make the compositions of the present disclosure. Various methods may also be utilized to administer the compositions of the present disclosure to subjects in order to treat various conditions or diseases.

Molecules

The molecules of the present disclosure can include, without limitation, riluzole, a derivative of riluzole, an analog of riluzole, a pharmaceutical equivalent of riluzole, a benzothiazole-based molecule, combinations thereof, and salts thereof. In some embodiments, the molecules of the present disclosure include riluzole (i.e., 6-trifluoromethoxy-2-benzothiazolamine) In some embodiments, the molecules of the present disclosure include a benzothiazole-based molecule, such as a benzothiazole class of a drug.

The molecules of the present disclosure may be in liquid formulations at various concentrations. For instance, in some embodiments, the molecules of the present disclosure may be in liquid formulations at concentrations ranging from 1 mg/ml to 25 mg/ml. In some embodiments, the molecules of the present disclosure may be in liquid formulations at concentrations of more than 5 mg/ml. In some embodiments, the molecules of the present disclosure may be in liquid formulations at concentrations ranging from 6 mg/ml to 25 mg/ml. In some embodiments, the molecules of the present disclosure may be in liquid formulations at a concentration of at least 7.5 mg/ml. In some embodiments, the molecules of the present disclosure may be in liquid formulations at a concentration of at least 10 mg/ml. In some embodiments, the molecules of the present disclosure may be in liquid formulations at a concentration of 10 mg/ml.

The molecules of the present disclosure may be associated with liquid formulations in various manners. For instance, in some embodiments, the molecules of the present disclosure are dissolved in the liquid formulation. In some embodiments, the molecules of the present disclosure are dispersed in the liquid formulation. In some embodiments, the molecules of the present disclosure are suspended in the liquid formulation. In some embodiments, the molecules of the present disclosure are dissolved and suspended in the liquid formulation.

Liquid Formulations

Liquid formulations generally refer to any liquids that can be associated with the molecules of the present disclosure. The liquid formulations of the present disclosure can have numerous components. For instance, in some embodiments, the liquid formulations of the present disclosure include without limitation, solubilizing agents, pharmaceutically acceptable carriers, excipients, syrups, elixir, water, gels, and combination thereof.

Solubilizing Agents

In some embodiments, the liquid formulations of the present disclosure include a solubilizing agent. Solubilizing agents generally refer to one or more compounds that are capable of facilitating the solubilization of the molecules of the present disclosure in liquid formulations. Solubilizing agents may also be referred to as co-solvents or carriers.

In some embodiments, the solubilizing agents of the present disclosure include water miscible organic solvents. In some embodiments, the solubilizing agents of the present disclosure include, without limitation, polyethylene glycol (e.g., PEG 400 and/or PEG 300), glycerin, propylene glycol, ethanol, sorbitol, polyoxyethylated glycerides (e.g., Labrafil M-2125CS), polyoxyethylated oleic glycerides (e.g., Labrafil M-1944CS, Polyoxyl 35 castor oil, and/or Cremophor EL), polysorbates (e.g., polysorbate 20 and/or polysorbate 80), sorbitan monooleate, hydroxypropyl-beta-cyclodextrin (HPCD), polyoxyl 40 hydrogenated castor oil (i.e., Cremophor RH 40), polyoxyl hydroxystearates (e.g., Solutol HS 15), and combinations thereof. In some embodiments, the solubilizing agents of the present disclosure include polyethylene glycol (e.g., PEG 400), glycerin, and propylene glycol.

The liquid formulations of the present disclosure may include various amounts of solubilizing agents. For instance, in some embodiments, the liquid formulations of the present disclosure contain from about 1% v/v to about 60% v/v of solubilizing agents. In some embodiments, the liquid formulations of the present disclosure contain from about 1% v/v to about 65% v/v of solubilizing agents. In some embodiments, the liquid formulations of the present disclosure contain from about 20% v/v to about 65% v/v of solubilizing agents. In some embodiments, the liquid formulations of the present disclosure contain less than about 70% v/v of solubilizing agents. In some embodiments, the liquid formulations of the present disclosure contain less than about 65% v/v of solubilizing agents. In some embodiments, the liquid formulations of the present disclosure contain less than about 60% v/v of solubilizing agents. In some embodiments, the liquid formulations of the present disclosure contain less than about 50% v/v of solubilizing agents.

In some embodiments, the liquid formulations of the present disclosure contain about 45% v/v of solubilizing agents. In some embodiments, the liquid formulations of the present disclosure contain about 15% v/v PEG 400, 20% v/v propylene glycol and 10% v/v of glycerin.

Pharmaceutically Acceptable Carriers

In various embodiments, the compositions and liquid formulations of the present disclosure can also be associated with one or more pharmaceutically acceptable carriers. Pharmaceutically acceptable carriers generally refer to a pharmaceutically acceptable material, composition, or vehicle that is involved in carrying or transporting the compositions of the present disclosure from one tissue, organ, or portion of the body to another tissue, organ, or portion of the body.

In some embodiments, the pharmaceutically acceptable carriers that are associated with the compositions and liquid formulations of the present disclosure may be a liquid or solid filler, diluent, excipient, solvent, encapsulating material, or a combination thereof. In some embodiments, each component of the pharmaceutically acceptable carrier must be compatible with the other ingredients of the compositions and liquid formulations of the present disclosure. In some embodiments, the pharmaceutically acceptable carrier must also be suitable for use in contact with any tissues or organs with which it may come in contact such that it does not carry a risk of toxicity, irritation, allergic response, immunogenicity, or any other complication that excessively outweighs its therapeutic benefits.

In some embodiments, a pharmaceutically acceptable carrier may include a pharmaceutically acceptable excipient. In some embodiments, the pharmaceutically acceptable excipient may be solid, liquid, semisolid, or gaseous. In some embodiments, a pharmaceutically acceptable excipient includes an excipient that is useful in preparing a pharmaceutical composition that is generally safe and non-toxic.

In some embodiments, the pharmaceutically acceptable carriers of the present disclosure may be added to enhance or stabilize the compositions of the present disclosure. In some embodiments, the pharmaceutically acceptable carriers of the present disclosure may be added to facilitate the preparation of the compositions of the present disclosure.

In some embodiments, the pharmaceutically acceptable carriers of the present disclosure may include liquid carriers. In some embodiments, the liquid carriers include, without limitation, syrup, peanut oil, olive oil, glycerin, saline, alcohols, water and combinations thereof.

In some embodiments, the pharmaceutically acceptable carriers of the present disclosure may include solid carriers. In some embodiments, solid carriers include, without limitation, starch, lactose, calcium sulfate, dihydrate, terra alba, magnesium stearate or stearic acid, talc, pectin, acacia, agar, gelatin, and combinations thereof.

In some embodiments, the pharmaceutically acceptable carriers of the present disclosure may also include a sustained release material. In some embodiments, the sustained release material includes, without limitation, glyceryl monostearate, glyceryl distearate, and combinations thereof. In some embodiments, the sustained release material may also be associated with a wax.

Liquid Formulation Properties

The liquid formulations of the present disclosure can have various advantageous properties. For instance, in some embodiments, the liquid formulations of the present disclosure have sufficient properties such that they are suitable for parenteral administration (e.g., intravenous, subcutaneous, intramuscular, or intra-articular administration). In some embodiments, the liquid formulations of the present disclosure have sufficient properties such that they are suitable for oral administration.

The liquid formulations of the present disclosure can have various pH values. For instance, in some embodiments, the liquid formulations of the present disclosure have pH values ranging from about 5 to about 9. In some embodiments, the liquid formulations of the present disclosure have pH values of about 7.

The liquid formulations of the present disclosure can provide the molecules of the present disclosure with various levels of stability. For instance, in some embodiments, the molecules in the liquid formulations of the present disclosure have a t₉₀ room temperature stability of more than 10 months. In some embodiments, the molecules in the liquid formulations of the present disclosure have a t₉₀ room temperature stability of more than 15 months. In some embodiments, the molecules in the liquid formulations of the present disclosure have a t₉₀ room temperature stability of more than 17 months.

In some embodiments, the molecules in the liquid formulations of the present disclosure have a 4° C. stability of more than 24 months. In some embodiments, the molecules in the liquid formulations of the present disclosure have a 4° C. stability of more than 30 months. In some embodiments, the molecules in the liquid formulations of the present disclosure have a 4° C. stability of more than 35 months.

The liquid formulations of the present disclosure can also have a high level of homogeneity. For instance, in some embodiments, the liquid formulations of the present disclosure represent a clear and homogenous solution. In some embodiments, the liquid formulations of the present disclosure represent a colorless solution at room temperature with no visible particles. In some embodiments, the liquid formulations of the present disclosure have a homogeneity sufficient for parenteral administration. In some embodiments, the liquid formulations of the present disclosure are in the form of a homogenous emulsion.

Methods of Treating a Condition or a Disease

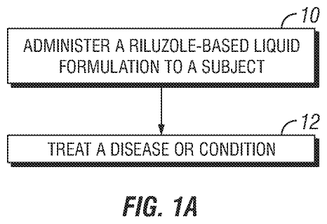

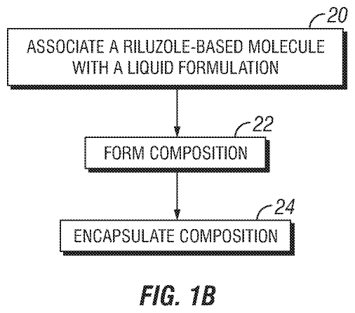

Additional embodiments of the present disclosure pertain to methods of treating a condition or disease in a subject. In some embodiments illustrated in FIG. 1A, the methods of the present disclosure comprise administering to the subject a composition of the present disclosure (step 10) in order to treat a disease or condition in the subject (step 12). In some embodiments, the compositions of the present disclosure are administered at a therapeutically effective dosage for treating a disease or condition. As set forth in more detail herein, the compositions of the present disclosure may be administered to various subjects in various manners in order to treat various diseases or conditions in the subject.

Subjects

The compositions of the present disclosure may be administered to various subjects. For instance, in some embodiments, the subject is a human being. In some embodiments, the subject is a non-human animal. In some embodiments, the non-human animal includes, without limitation, mice, rats, rodents, mammals, cats, dogs, monkeys, pigs, cattle and horses. In some embodiments, the subject is a rat. In some embodiments, the subject is suffering from a condition or disease to be treated by the methods of the present disclosure.

Administration

The compositions of the present disclosure may be administered to subjects through various routes. For instance, in some embodiments, the compositions of the present disclosure may be administered through administration routes that include, without limitation, oral administration, inhalation, subcutaneous administration, intravenous administration, intraperitoneal administration, intramuscular administration, intrathecal injection, intra-articular administration, topical administration, central administration, peripheral administration, aerosol-based administration, nasal administration, transmucosal administration, transdermal administration, parenteral administration, and combinations thereof.

In some embodiments, the administration occurs by intravenous administration. In some embodiments, the administration occurs by oral administration. In some embodiments, the administration occurs by parenteral administration.

Parenteral administration generally refers to a route of administration that is generally associated with injection. In some embodiments, the parenteral administration includes, without limitation, intraorbital administration, infusion, intraarterial administration, intracapsular administration, intracardiac administration, intradermal administration, intramuscular administration, intraperitoneal administration, intrapulmonary administration, intraspinal administration, intrasternal administration, intrathecal administration, intrauterine administration, intravenous administration, subarachnoid administration, subcapsular administration, subcutaneous administration, transmucosal administration, transtracheal administration, intra-articular administration, and combinations thereof.

In some embodiments, the compositions of the present disclosure may be administered in a therapeutically effective amount. In some embodiments, the therapeutically effective amount is that amount of the composition that will yield the most effective results in terms of efficacy of treatment in a given subject. In some embodiments, this amount will vary depending upon a variety of factors, including, but not limited to, the characteristics of the compositions of the present disclosure (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a composition and adjusting the dosage accordingly. For additional guidance, see Remington: The Science and Practice of Pharmacy (Gennaro ed. 20th edition, Williams & Wilkins Pa., USA) (2000).

Conditions or Diseases

The compositions of the present disclosure can be utilized to treat various conditions or diseases in a subject. For instance, in some embodiments, the condition or disease to be treated include, without limitation, spinal cord injury (SCI), swallowing abnormalities, dysphagia, a neurological disease or condition, amyotrophic lateral sclerosis (ALS), and combinations thereof. In some embodiments, the condition or disease to be treated includes spinal cord injury, such as acute spinal cord injury. In some embodiments, the condition or disease to be treated includes a swallowing abnormality, such as dysphagia.

Effects

The administered molecules in the compositions of the present disclosure can demonstrate fast absorption rates in subjects after administration. For instance, in some embodiments, the administered molecules of the present disclosure demonstrate an absorption half-life of less than 1.5 hours. In some embodiments, the administered molecules of the present disclosure demonstrate an absorption half-life of less than 1 hour. In some embodiments, the administered molecules of the present disclosure demonstrate an absorption half-life of less than 0.5 hours. In some embodiments, the administered molecules of the present disclosure demonstrate an absorption half-life of less than 0.3 hours.

The administered molecules in the compositions of the present disclosure can also demonstrate sustained bioavailability in subjects after administration. For instance, in some embodiments, the administered molecules of the present disclosure demonstrate an elimination half-life of more than 10 hours. In some embodiments, the administered molecules of the present disclosure demonstrate an elimination half-life of more than 15 hours. In some embodiments, the administered molecules of the present disclosure demonstrate an elimination half-life of more than 20 hours. In some embodiments, the administered molecules of the present disclosure demonstrate an elimination half-life of at least 10 hours.

In some embodiments, the administered molecules of the present disclosure demonstrate a bioavailability of more than 65%. In some embodiments, the administered molecules of the present disclosure demonstrate a bioavailability of more than 70%. In some embodiments, the administered molecules of the present disclosure demonstrate a bioavailability of more than 75%. In some embodiments, the administered molecules of the present disclosure demonstrate a bioavailability of more than 80%. In some embodiments, the administered molecules of the present disclosure demonstrate a bioavailability of more than 85%. In some embodiments, the administered molecules of the present disclosure demonstrate a bioavailability of more than 90%.

In some embodiments, the administered molecules in the compositions of the present disclosure can become localized in the central nervous system. For instance, in some embodiments, the brain (μg/g) to plasma (μg/ml) ratio of the administered molecule is more than 3.5 after 24 hours of administration. In some embodiments, the brain (μg/g) to plasma (μg/ml) ratio of the administered molecule ranges from about 2 to about 6 after 24 hours of administration. In some embodiments, the brain (μg/g) to plasma (μg/ml) ratio of the administered molecule ranges from about 3.5 to about 5 after 24 hours of administration.

In some embodiments, the spinal cord (μg/g) to plasma (μg/ml) ratio of the administered molecule is more than 6.5 after 24 hours of administration. In some embodiments, the spinal cord (μg/g) to plasma (μg/ml) ratio of the administered molecule ranges from about 5 to about 10 after 24 hours of administration. In some embodiments, the spinal cord (μg/g) to plasma (μg/ml) ratio of the administered molecule ranges from about 6.5 to about 10 after 24 hours of administration.

In some embodiments, the compositions of the present disclosure can substantially eliminate the symptoms associated with a disease or condition to be treated. In some embodiments, the compositions of the present disclosure can treat a disease or condition in a subject without showing substantial hemolysis. For instance, in some embodiments, the compositions of the present disclosure may show less than 20% or less than 15% hemolysis in a subject after administration in an F/B range of 0.005 to 0.01. In some embodiments, the compositions of the present disclosure demonstrate an in vitro hemolysis of less than 10% in the F/B range of 0.005 to 0.01.

Methods of Making the Compositions

Additional embodiments of the present disclosure pertain to methods of making the compositions of the present disclosure. In some embodiments illustrated in FIG. 1B, the methods of the present disclosure include a step of associating a molecule of the present disclosure with a liquid formulation (step 20) to form the compositions of the present disclosure (step 22).

Various methods may be utilized to associate the molecules of the present disclosure with liquid formulations. For instance, in some embodiments, the associating occurs by a method that includes, without limitation, mixing, stirring, heating, milling, compressing, and combinations thereof. In some embodiments, the association occurs by mixing.

In some embodiments illustrated in FIG. 1B, the methods of the present disclosure also include a step of encapsulating the formed compositions in a carrier (step 24). In some embodiments, the carrier is a capsule, such as a soft gelatin capsule.

Kits

Additional embodiments of the present disclosure pertain to kits that contain the compositions of the present disclosure. In some embodiments, the kit also includes one or more materials for administering the compositions of the present disclosure to a subject. In some embodiments, the one or more materials are suitable for parenteral administration of the compositions of the present disclosure to a subject. In some embodiments, the one or more materials are suitable for intravenous administration of the compositions of the present disclosure to a subject.

In some embodiments, the compositions in the kit include a composition that includes a parenteral preparation of riluzole. In some embodiments, the compositions in the kit include a composition that includes an intravenous preparation of riluzole.

In some embodiments, the exact nature of the components configured in the kits of the present disclosure depend on the kit's intended purpose. For instance, in some embodiments, the kits of the present disclosure are configured for the purpose of treating a neurological condition or disease, such as acute spinal cord injury. In some embodiments, the kits of the present disclosure are configured for the purpose of treating mammalian subjects. In some embodiments, the kits of the present disclosure are configured for the purpose of treating human subjects. In some embodiments, the kits of the present disclosure are configured for veterinary applications, such as treating farm animals, domestic animals, and laboratory animals.

In some embodiments, the kits of the present disclosure may also include instructions for using the kits. In some embodiments, the instructions may include a tangible medium of expression describing the technique to be employed in using the components of the kit to effect a desired outcome, such as to prepare or administer a liquid formulation of riluzole.

In some embodiments, the kits of the present disclosure may also include additional components. In some embodiments, the additional components may include, without limitation, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kits of the present disclosure can be provided to a practitioner stored in any convenient and suitable ways that preserve their operability and utility. For instance, in some embodiments, the components can be in dissolved, dehydrated, or lyophilized form. In some embodiments, the components can be provided at room, refrigerated or frozen temperatures.

In some embodiments, the components can be contained in suitable packaging material(s). As used herein, a packaging material refers to one or more physical structures used to house the contents of a kit, such as the compositions of the present disclosure. In some embodiments, the packaging material may be constructed by well-known methods, such as methods that preferably provide a sterile, contaminant-free environment.

In some embodiments, a packaging material can include a suitable solid matrix or material such as glass, plastic, paper, foil, and the like. In some embodiments, the packaging material is capable of holding individual kit components. In some embodiments, the packaging material can be a glass vial used to contain suitable quantities of the compositions of the present disclosure (e.g., liquid formulations of riluzole). In some embodiments, the packaging material has an external label which indicates the contents and purpose of the kit and its components.

Applications and Advantages

The methods, kits, and compositions of the present disclosure can have various advantages. For instance, due to their liquid form, the compositions of the present disclosure can be administered to subjects in a more facile manner and with the capability to adjust the dosage of molecules in the composition. Moreover, in some embodiments, the molecules of the present disclosure can be solubilized in liquid formulations at high concentrations through the use of solubilizing agents, such as polyethylene glycol, propylene glycol and glycerin.

Furthermore, the molecules in the compositions of the present disclosure can show optimum stability at different temperatures (e.g., from room temperature to 4° C.) for prolonged periods of time (e.g., from 17 to 35 months). In addition, the compositions of the present disclosure can be suitable for different modes of administration, such as oral and intravenous administration.

As such, when compared to commercial tablets, the molecules in the compositions of the present disclosure demonstrate faster rates of absorption and more sustained plasma levels with a significantly longer elimination half-life. Moreover, due to optimal stability, ease of administration, consistent dosing, rapid onset and longer duration of action, the molecules in the compositions of the present disclosure can be beneficial for the treatment of various diseases and conditions, such as acute spinal cord injury (SCI). In addition, due to ease of administration, the molecules in the compositions of the present disclosure can be utilized to treat various patients with limited ability for the ingestion of drugs, such as SCI patients.

Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### EXAMPLE 1

### Development and Characterization of Stable Aqueous Liquid Formulations of Riluzole

In this Example, Applicants aimed to develop a stable aqueous liquid formulation of riluzole for administration in a spinal cord injury (SCI) population and with individualized dose if needed. Since riluzole has poor water solubility, solubilization using co-solvents was developed to develop an aqueous based liquid formulation, based on a Center Composite Design (CCD) approach. The most commonly used co-solvents were used, such as, for example, propylene glycol, Polyethylene Glycol 400 (PEG 400) and glycerin to develop the formulation.

An optimum liquid formulation of riluzole was successfully formulated and consisted of 15%, 20% and 10% v/v of PEG 400, propylene glycol and glycerin, respectively, with a riluzole concentration of 10 mg/ml. The optimum composition was characterized and assessed for stability at different temperatures. Satisfactory stability was obtained at room temperature and 4° C. (t₉₀ of 17 and 35 months, respectively).

In vitro hemolysis studies were performed on whole rat blood. The formulation was non hemolytic at the intended dose level. The optimum formulation of riluzole was suitable for both oral and intravenous administrations. Single dose pharmacokinetic studies of the optimum formulation were evaluated in male Sprague Dawley rats, by both oral and IV routes. The formulation was well tolerated and the pharmacokinetics was characterized by both routes.

### EXAMPLE 1.1

### Rational Design with Center Composite Design (CCD)

Since riluzole has poor water solubility, solubilization using co-solvents was employed to develop the aqueous solution that will provide consistent dose with ease of administration. Co-solvency is one of the most common and popular approaches to solubilize poorly water-soluble drugs. Co-solvents reduce the polarity of water by weakening its intermolecular hydrogen-bonding network and thereby increasing the aqueous solubility of non-polar drugs. The most commonly used co-solvents are propylene glycol, PEG 400, glycerin and ethanol. A large number of FDA approved marketed oral solution formulations contain these co-solvents, either alone or as mixtures.

A systematic design of experimental approach was utilized to determine the optimum composition that can solubilize the entire 50 mg dose of riluzole, with minimum concentrations of co-solvents. Statistical designs such as response surface designs provide useful information on direct effects, pairwise interaction effects and curvilinear effects of different variables. One of the most popular response surface designs is the Central Composite Design (CCD) and has been widely used for optimization purposes. Several types of CCD are available that can be selected based on the experimental regions of interest and operability. Furthermore, these designs are efficient in providing information on effects of experimental variables and overall experimental error in a minimum number of experimental runs.

In this Example, Applicants utilized CCD to identify the optimal concentrations of co-solvents which significantly impacted riluzole solubility, and developed a stable, aqueous based solution formulation of riluzole. In addition, CCD was used to predict the amount of riluzole dissolved in a given composition of co-solvents, using an established and validated model. The optimum formulation was subsequently evaluated for in vivo disposition in rats after oral and IV administration.

### EXAMPLE 1.2

### Materials

Riluzole (RIL) was purchased from Tecoland Corporation (Irvine, USA). Salts used to prepare buffer solutions were purchased from Sigma Aldrich (St. Louis, Mo., USA). Polyethylene glycol 400 (PEG 400), propylene glycol (PG) and glycerin (GLY) were purchased from Avantor Performance Materials Inc. (Center Valley, Pa., USA). Ora-Plus Oral suspending vehicle was purchased from Perrigo Company Inc. (Allegan, Mich., USA). Deionized ultrapure water was purified using Milli-Q Water Purification System (Bedford, Mass., USA). Membrane syringe PTFE filters (25 mm, 0.45 μm) were procured from VWR International (Radnor, Pa., USA). 5-Methoxy psoralen, used as internal standard for HPLC analysis, was purchased from Sigma Aldrich (St. Louis, Mo., USA). All solvents (Acetonitrile, Methanol) used for chromatographic analysis were of HPLC grade and purchased from VWR (Radnor, Pa., USA). Whole rat blood (pooled) was purchased from Biochemed Services (Winchester, Va., USA). Rilutek tablets (manufactured by Rising Pharmaceuticals, Allendale, N.J., USA) were provided by Houston Methodist Hospital (Houston, Tex., USA).

### EXAMPLE 1.3

### Animals

Male Sprague Dawley rats (250-300 g) were purchased from Envigo (Indianapolis, Ind., USA). For intravenous administration of formulation, male Sprague Dawley rats with jugular vein cannulation were also purchased from Envigo (Indianapolis, Ind., USA). The animals were allowed access to food and water, and placed in an environmentally controlled room (temperature: 25±2° C., humidity: 50±5%, 12 hr dark-light cycle). The animal protocol was approved by the Institutional Animal Care and Use Committee (IACUC) of University of Houston.

### EXAMPLE 1.4

### HPLC Analysis of Riluzole

Chromatographic Conditions: The HPLC method used to quantify riluzole has been described previously. See Chow et al, Journal of neurosurgery, Spine 17, 129-140. The assay was performed using a Waters HPLC system equipped with 717 plus autosampler, 515 HPLC pump and 2996 UV detector. In brief, chromatographic separation was achieved using a Waters Symmetry C18 column (3.0×150 mm, 3.5 μm) with Symmetry C18 guard column (2.1×10 mm, 3.5 μm). The mobile phase consisted of acetonitrile, methanol and 0.1 M ammonium acetate in the ratio of 3:2:5, v/v/v, adjusted with acetic acid to pH 6.5. The flow-rate was set at 0.45 ml/min and the injection volume was 50 μL. Riluzole was detected at 263 nm.

Preparation of Stock and Standard Solutions: Stock solutions of riluzole and the internal standard (5-Methoxy psoralen, 5-MOP) were prepared in methanol at a concentration of 1 mg/ml. Standard working solutions of riluzole (10 μg/ml) and 5-MOP (50 μg/ml) were prepared in methanol-water (50:50, v/v) solution. Standard solutions of riluzole were prepared by serial dilutions of the stock solution, in the linearity range of 7.8 to 1,000 ng/ml. Quality control samples (31.25, 125 and 1,000 ng/ml) were also run along with the samples to determine the accuracy and precision in each run.

Quantification of Riluzole in Formulation: 50 μl of formulation was diluted in the mobile phase to determine riluzole content. All samples were analyzed in triplicates.

Quantification of Riluzole in Rat Plasma: An eight point calibration curve was prepared in the range of 7.8-1,000 ng/ml. One hundred (100) μL of blank plasma was spiked with 10 μL of internal standard (50 μg/ml of 5-MOP) and 10 μL of drug standard solutions to prepare the calibration curve standards. The riluzole samples were vortexed for 30 seconds, and extracted from plasma using 500 μL of ethyl acetate. The samples were vortexed and centrifuged at 17,968×g for 20 minutes at 4° C. The supernatant was aliquoted, air dried, and reconstituted with 1 ml of mobile phase, vortexed and centrifuged. The samples were then analyzed by the HPLC assay method. The HPLC method was validated as per FDA Guidance.

### EXAMPLE 1.5

### Formulation Development

Experimental Design: A three-factor, five-level rotatable design (α=1.68) was selected and a full CCD matrix with 20 compositions was prepared. Three different co-solvents (PEG 400, PG and GLY) were used in order to minimize the concentrations of individual co-solvents in the composition. These co-solvents have been extensively used in FDA approved drug products for different routes of administration. In one embodiment, Applicants identified from the Inactive Ingredients Database of FDA and other literature sources the approved concentrations for these co-solvents for oral and IV routes of administration. The concentration range to be evaluated for each solvent was then evaluated.

Each co-solvent (factor) was evaluated at five concentration levels. The factors and the corresponding levels (coded and actual values) were compiled in Table 1. To get a better understanding of the effects of co-solvents, a full CCD with 20 runs was employed, which consisted of 6 center points, 6 axial points and 8 factorial or edge points. The center point is repeated several times in the design in order to increase the confidence in the measured response values.

The 20 compositions were prepared in three blocks (three days) and each composition was prepared in triplicates and analyzed by the HPLC method for riluzole content. The CCD design enabled Applicants to evaluate the main effects and the interactive effects of the co-solvent ingredients used to prepare the formulations. The value of α was selected at 1.682, which provides rotatability to the design. A rotatable design allows uniformity of prediction error at all points. Design-Expert software (version 9.0.3; Stat-Ease, Inc., Minneapolis, Minn.) was used to generate the design, analyze the results, and perform the optimization.

| Co-solvents | Concentration (% v/v) (Levels) | | | | |
|---|---|---|---|---|---|
| (Factors) | −α | −1 | 0 | +1 | +α |
| Polyethylene Glycol 400 (PEG 400) | 1.2 | 6 | 13 | 20 | 24.8 |
| Propylene glycol (PG) | 3.2 | 10 | 20 | 30 | 36.8 |
| Glycerin (GLY) | 1.6 | 5 | 10 | 15 | 18.4 |

Table 1: Factors (co-solvents) and their levels evaluated using Central Composite Design.

The central values were coded as 0. The factorial or edge points (−1 and +1) and the axial (−α and +α) points augment the entire design. The amount of riluzole dissolved in a given composition was the chosen response variable. The experimental data were compared by fitting into various mathematical models. The model that best described the data was then selected based on various parameters, such as p values, the multiple correlation coefficients (R²), predicted multiple correlation coefficients (predicted R²), adjusted multiple correlation coefficients (adjusted R²) and the predicted residual sum of squares. Analysis of variation (ANOVA) was performed to determine if the factors and the interactions between the factors were statistically significant. The chosen model was defined by a polynomial regression equation which describes the relationship between the response (dependent) and the independent variables and considers the linear terms, the square terms and the interaction of the linear terms as provided in Equation 1 herein:

Y=b₀+Σ bᵢXᵢ+Σ bᵢᵢXᵢᵢ Σ bᵢⱼXᵢXⱼ+ε Eq. 1

In Equation 1, b₀ is a constant; bᵢ is the slope or linear effect of the input factor Xi; bij is the linear interaction effect between the input factors Xᵢ and X_{j;} and bᵢᵢ is the quadratic effect of the input factor Xᵢ and Y is the response.

The established model was validated at three different levels of the response variable to prove the accuracy and usefulness of the selected model in predicting and optimizing the composition. Inter and intra-day accuracy and precision were determined at the three response levels with n=3 at each level.

Evaluation of Formulations: All co-solvent formulations prepared were analyzed for riluzole content by the HPLC assay method. The optimized formulation was evaluated for color, appearance, pH, in vitro hemolytic potential, stability at different temperatures, and in vivo plasma pharmacokinetics after oral and IV administrations.

Stability Studies: The optimized co-solvent formulation was filled into amber glass vials and stored for 3 months. The stability was assessed at 4° C., 25° C., 37° C. and 60° C. The samples were withdrawn at predetermined intervals (individual vials at each time point) and evaluated for physical (drug precipitation and clarity) and chemical (drug content by HPLC) stability. The rate constant of degradation at each temperature was derived, and the shelf life at room temperature was predicted using Arrhenius plot.

In Vitro Hemolytic Potential: The hemolytic potential of riluzole co-solvent formulation was determined using a modified method previously published. See Nornoo et al, International Journal of Pharmaceutics, 349, 108-116 (2008).

Volume of Water to Produce Complete Hemolysis: Different volumes of distilled water were added to 0.2 ml of heparinized pooled whole rat blood to obtain various ratios of water/blood (0.1, 0.2, 0.5, 1, 5 and 10). The mixtures were mixed for 10 s, incubated for 2 min at room temperature, and 5 ml of saline was added. The solutions were centrifuged (Eppendorf Centrifuge 5810 R) at 1,500×g for 5 minutes at 25° C. The supernatant was discarded and the packed cells were washed with 5 ml of saline and again centrifuged at 1500×g for 5 minutes. The supernatant was discarded and intact cells were lysed with 1 ml water. The samples were diluted with distilled water at 1:9 ratio and absorbance was measured at 540 nm using a DU 800 Spectrophotometer (Beckmann, Fullerton, USA). The water/blood ratio that produced insignificant absorbance was identified as the ratio at which complete hemolysis occurs. This solution with complete hemolysis (Solution A) was further used to generate a standard curve.

Standard Curve and Sample Preparation: Different ratios of healthy RBC cell fraction (0, 0.2, 0.5, 0.8 and 1) were prepared. This was done by mixing Solution A and blood in different ratios to a total volume of 1 ml. The mixtures were vortexed for 2-3 seconds, and equilibrated for 2 min at room temperature. Five (5) ml of saline was added to stop the hemolysis. The solutions were centrifuged at 1,500×g for 5 minutes at 25° C. A standard curve of absorbance versus ratio of healthy cell fraction was constructed that was used later to determine the healthy cell fraction in the formulation spiked blood samples.

For evaluating the hemolytic potential of the developed formulation, various volumes of formulation were mixed individually with whole rat blood to obtain formulation to blood (F/B) ratios in the range of 0.025 to 2. The procedure followed was the same as in the standard curve protocol. The fraction of healthy cells was calculated from the standard curve. The hemolytic potential was derived from the plot of fractions of healthy cells versus the formulation/blood ratios.

Pharmacokinetic Study: Male Sprague Dawley rats (250-300 grams) were used to evaluate the in vivo plasma pharmacokinetics of the developed co-solvent formulation after a single dose of oral and intravenous administrations. In addition, plasma PK of crushed commercial tablet, Rilutek (Sanofi) was evaluated after a single oral administration, as a reference.

Based on the clinical dose of 100 mg per day and LD₅₀ reported in product monograph (oral: 45 mg/kg and intravenous: 21 mg/kg in rats), the oral dose of 10 mg/kg and intravenous (IV) dose of 5 mg/kg were derived using allometric scaling, for animal study.

All animals were divided into three groups (n=5-7 each group). The first group received Rilutek tablets (crushed and suspended) orally at a dose of 10 mg/kg. The tablets were crushed and weighed based on the calculated dose in each animal. The crushed powder was then suspended in a commercial suspending vehicle, Ora-Plus.

The second and third groups received the developed liquid formulation orally (10 mg/kg) and intravenously (5 mg/kg). Blood samples (approx. 0.2 ml) were taken via tail vein at 15 min, 30 min, 1, 3, 6, 9, 12 and 24 hr post dose. The blood samples were centrifuged at 5,867×g for 10 minutes at 4° C. Thereafter, the plasma was collected and stored at −80° C. until analysis. The samples were analyzed by the validated HPLC method to quantify riluzole concentrations.

Pharmacokinetic Data Analysis: The pharmacokinetic parameters were derived from the concentration time profiles using compartmental model of Phoenix WinNonLin 6.3. Student-t test was performed on the PK parameters to determine any statistical significance (p≤0.05) between the different formulations (Co-solvent vs. crushed tablet) and routes of administration (oral vs IV).

### EXAMPLE 1.6

### Liquid Formulations of Riluzole

HPLC Assay of Riluzole: A validated HPLC method was used for riluzole quantification from rat plasma samples. The retention time for riluzole and 5-MOP (internal standard) were 9.0 and 7.4 min, respectively. No interfering peaks were observed for either riluzole or the internal standard. The linearity was observed over the concentration range of 7.8 (LLOQ)-1,000 ng/ml with a coefficient of correlation greater than 0.999. The recovery of riluzole from rat plasma ranged from 92.7-98.9%. The assay accuracy and precision were in the ranges of 94.6-103.4% and 1.6-2.8% (n=15), respectively, in rat plasma. The method was successfully used to determine riluzole concentrations in all plasma samples from the PK studies.

Analysis of Central Composite Design: One objective was to develop an aqueous based liquid formulation for riluzole that could be administered with ease in spinal cord injured patients. Riluzole is lipophilic and has low aqueous solubility. In one embodiment, Applicants measured the riluzole solubility at pH 5-9 and water (pH 7), 0.3 mg/ml, which is in agreement with the reported values.

No aqueous solution formulation of riluzole is currently available. Therefore, the aqueous solubility was enhanced using water miscible organic solvents, namely, PEG 400, propylene glycol (PG) and glycerin (GLY) to formulate a liquid solution containing 50 mg of riluzole in 5 ml of solvent mixture of volume, a 33-time enhancement.

The full CCD design matrix with 20 compositions were evaluated to determine the amount of riluzole solubilized in 5 ml of each given composition (Table 2).

TABLE 2
Design Space of Central Composite Design and Measured
Response Values (Mean ± SD, n = 3). Each composition
was prepared in triplicate.

| | | | | | Amount of |
|---|---|---|---|---|---|
| | | Conc. (% v/v) | | | Riluzole (mg) |
| Design | Type | PEG 400 | PG | GLY | Dissolved in 5 mL |
| (+1, −1, +1) | Edge | 20 | 10 | 15 | 50.77 ± 1.36 |
| (−1, −1, −1) | Edge | 6 | 10 | 5 | 8.78 ± 1.89 |
| (−1, +1, +1) | Edge | 6 | 30 | 15 | 51.46 ± 2.88 |
| (0, 0, 0) | Center | 13 | 20 | 10 | 43.25 ± 4.37 |
| (0, 0, 0) | Center | 13 | 20 | 10 | 48.38 ± 4.51 |
| (+1, +1, −1) | Edge | 20 | 30 | 5 | 52.23 ± 2.07 |
| (+1, -1, −1) | Edge | 20 | 10 | 5 | 48.30 ± 1.18 |
| (0, 0, 0) | Center | 13 | 20 | 10 | 47.39 ± 1.02 |
| (0, 0, 0) | Center | 13 | 20 | 10 | 48.52 ± 5.34 |
| (+1, +1, +1) | Edge | 20 | 30 | 15 | 51.85 ± 3.23 |
| (−1, −1, +1) | Edge | 6 | 10 | 15 | 11.75 ± 0.79 |
| (−1, +1, −1) | Edge | 6 | 30 | 5 | 47.48 ± 1.52 |
| (−α, 0, 0) | Axial | 1.2 | 20 | 10 | 11.43 ± 3.16 |
| (0, 0, −α) | Axial | 13 | 20 | 1.6 | 36.28 ± 7.56 |
| (0, 0, 0) | Center | 13 | 20 | 10 | 44.32 ± 6.51 |
| (0, +α, 0) | Axial | 13 | 36.8 | 10 | 54.18 ± 5.06 |
| (0, −α, 0) | Axial | 13 | 3.2 | 10 | 14.77 ± 1.94 |
| (+α, 0, 0) | Axial | 24.8 | 20 | 10 | 50.88 ± 1.75 |
| (0, 0, +α) | Axial | 13 | 20 | 18.4 | 53.24 ± 2.35 |
| (0, 0, 0) | Center | 13 | 20 | 10 | 49.20 ± 3.74 |

The amount of riluzole solubilized (in 5 ml) in these 20 unique compositions ranged from 8.8 mg to 54.2 mg. In the instant study, the entire design has 6 replicates of central points (0, 0, 0) to improve the predictions and provide statistically sound results. The composition with 13%, 20% and 10% v/v of PEG 400, PG and GLY, respectively, represented the center point of the design and it solubilized 46.85±2.46 mg of riluzole in 5 ml. The observed solubilities were fitted into different models of increasing polynomial complexity to identify the model that best described the observed data. The sequential sum of squares showed that when quadratic (squared) terms were added to the mean, block, linear and two factor interaction terms, the model improved significantly with prob >F value less than 0.05. The fit summary (Table 3a) shows that quadratic model has the lowest standard deviation (4.89), highest R squared (0.9222) and lowest PRESS values among the tested models. In addition, the lack of fit test was insignificant (prob >F greater than 0.05) for the quadratic model, confirming that it is the best fit model for the instant observations.

TABLE 3a
Summary of Statistics used to select the model.

| Model Summary Statistics | | | | | |
|---|---|---|---|---|---|
| | Standard | | | Lack of Fit tests | |
| Source | Deviation | R squared | PRESS | F Value | Prob > F |
| Linear | 9.10 | 0.6965 | 5586.15 | 3.01 | 0.0122 |
| Two Factor | 6.91 | | | | |
| Interaction | | 0.8344 | 3032.98 | 1.59 | 0.1677 |
| 2FI | | | | | |
| Quadratic | 4.89 | 0.9222 | 1728.97 | 0.58 | 0.9087 |

ANOVA was performed for the selected quadratic model to determine the individual and interactive effects of the independent variables (concentration of each co-solvent) on the dependent response variable. The results are tabulated in Table 3(b).

TABLE 3b
Summary of Analysis of Variance (ANOVA)

| | Sum of | | | p-value |
|---|---|---|---|---|
| Source | Squares | Mean Square | F- Value | (Prob > F) |
| Block | 16.93 | 8.47 | | |
| Model | 13579.79 | 1508.87 | 63.20 | <0.0001 |
| A-PEG 400 | 4943.19 | 4943.19 | 207.04 | <0.0001 |
| B-Propylene | 4923.30 | 4923.30 | 206.21 | <0.0001 |
| Glycol | | | | |
| C-Glycerin | 390.39 | 390.39 | 16.35 | 0.0002 |
| AB | 2020.33 | 2020.33 | 84.62 | <0.0001 |
| AC | 8.82 | 8.82 | 0.37 | 0.5462 |
| BC | 1.27 | 1.27 | 0.053 | 0.8183 |
| A\hat{ }2 | 903.98 | 903.98 | 37.86 | <0.0001 |
| B\hat{ }2 | 499.36 | 499.36 | 20.92 | <0.0001 |
| C\hat{ }2 | 2.60 | 2.60 | 0.11 | 0.7429 |
| Residual | 1146.01 | 23.88 | | |
| Lack of Fit | 640.32 | 19.40 | 0.58 | 0.9087 |
| Pure Error | 505.69 | 33.71 | | |

The results confirmed that the adequacy of the selected model (Model Prob >F) is less than 0.05. Moreover, probability values of individual and interactive terms showed that the individual concentrations of PEG 400 and PG, as well as the interaction between concentrations of PEG 400 and PG, have significant effects on the amount of riluzole dissolved (Prob>F is less than 0.0001). Coefficients for each of the terms was generated, which indicate the extent of contribution of each term towards the overall observed response.

An equation with quadratic model coefficients derived, which defines the relationship between dependent variable and independent variables (direct and interactive), can be utilized to predict responses with given variables. The quadratic equation is as follows: Amount of riluzole dissolved (mg in 5 ml)=46.70+10.98*A+10.96*B+3.09*C−9.18*AB−0.61*AC−0.23*BC−4.57*A²−3.40*B². In this equation, A, B and C represent PEG 400, PG and GLY, respectively.

Diagnostic plots were checked to evaluate the residual assumptions of the analysis of variance. The normal probability plot of residuals is almost linear, indicating a normal distribution of residuals. The plot of residuals versus experimental run order has a random scatter, which rules out the possibility of any extraneous variable influencing the response during the experiment.

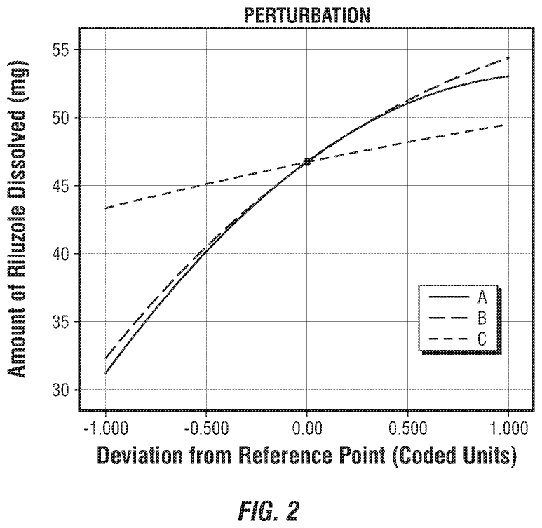

The perturbation plot (FIG. 2) was evaluated, which displayed the effect of each factor on the response, and it revealed that concentrations of PEG 400 and PG had significant effects, whereas that of GLY has very weak influence on the solubilization of riluzole. Therefore, the GLY concentration was fixed at 10% v/v of the center point.

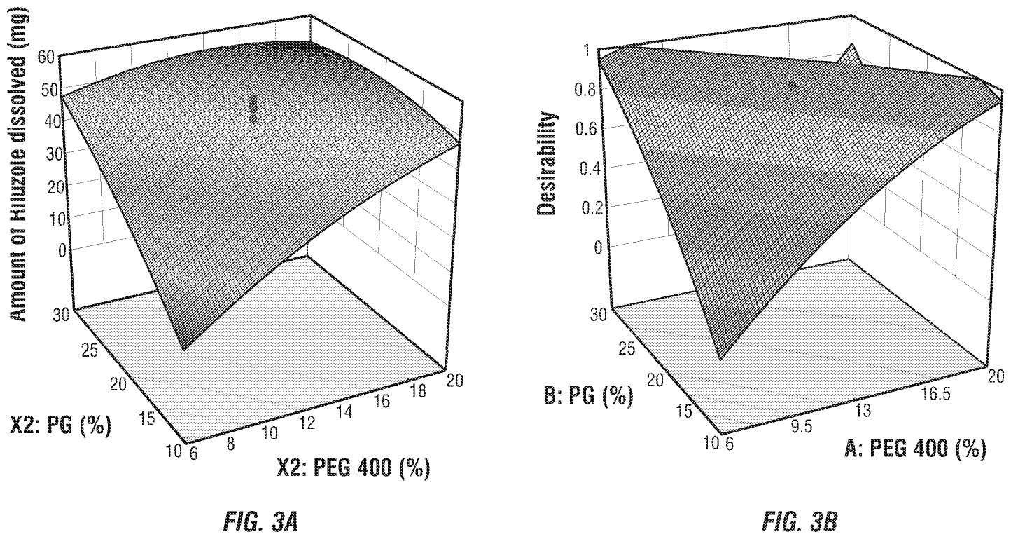

The 3D response surface plot (FIG. 3A) was constructed to show the relationship between the concentrations of PEG 400 and PG, and the resulting amount of riluzole dissolved (in 5 ml). The 3D surface plot enables Applicants to establish an appropriate value of the independent factors (concentration of individual components of co-solvents) to achieve the desired amount of dissolved riluzole.

Prior to determining the final optimum composition, in one embodiment, Applicants validated the established model at different levels of the response variable as shown in Table 4.

The established model was successfully validated. The intra-day and inter-day accuracy and precision values were satisfactory and provide confidence that the model can predict the response value for a given composition and vice versa. The intra- and inter-day accuracy values range from 98.7 to 105.8% and 100.8-101.6%, respectively, and the precision values range from 0.64 to 4.38% (less than 5% RSD).

TABLE 4
Validation of the Selected Model. The predicted amount of dissolved riluzole was obtained from
the Design Expert software. The observed amount of riluzole was determined experimentally using
the composition defined from the software. Accuracy was calculated as the percentage of observed
value over predicted value. Precision was reported as % relative standard deviation (%RSD).

| Amount of | | Intra Day (n = 3) | | | Inter Day (n = 6) | | |
|---|---|---|---|---|---|---|---|
| Riluzole (mg) | | Observ | Accuracy (%) | Precision | | Accuracy (%) | Precision |
| Desired | Predicted | ed | (Mean ± SD) | (% RSD) | Observed | (Mean ± SD) | (% RSD) |
| 10 | 10.1 | 10.2 ± 0.4 | 101.4 ± 4.4 | 4.38 | 10.2 ± 0.4 | 100.8 ± 4.0 | 3.94 |
| 50 | 49.9 | 52.9 ± 0.3 | 105.8 ± 0.7 | 0.64 | 50.8 ± 1.6 | 101.6 ± 3.3 | 3.21 |
| 55 | 55.1 | 54.4 ± 1.0 | 98.7 ± 1.8 | 1.84 | 55.7 ± 2.4 | 101.3 ± 4.3 | 4.25 |

The optimization module of the Design Expert software was used to select the optimum composition for a specified target response, utilizing the quadratic model. The minimum concentrations of PEG 400 and PG required to solubilize 50 mg of riluzole in 5 ml was estimated to be 15% and 20% v/v, respectively, with a desirability of 0.8 (shown in FIG. 3B). Therefore, it was concluded that the final optimum composition was 15%, 20% and 10% v/v of PEG 400, PG and GLY, respectively. The concentrations of individual co-solvents in the final composition were within the acceptable limits for both oral and intravenous administrations, based on their uses in FDA approved commercial formulations.

Evaluation of Optimum Formulation: The optimized co-solvent formulation of riluzole was evaluated for appearance, color, pH, drug content, stability and in vitro hemolytic potential. The freshly prepared formulation was a clear solution, with pH of 6.3, and no visible particles. The assayed contents of riluzole were in the range of 98-101% of 50 mg/5 ml.

Stability of Optimum Formulation: The physical and chemical stability of the optimum formulation was evaluated for 3 months at 4° C., 25° C., 37° C. and 60° C. Physical properties of the liquid formulation, such as appearance and color, were analyzed visually before the start of the chemical stability studies, and at every sampling time point when the drug content was monitored. The initial appearance of the formulation was a clear colorless liquid without any visible particles. After 3 months, the formulation remained clear and colorless at all temperatures, except at 60° C. Formulation kept at 60° C. showed opacity with some visible particles.

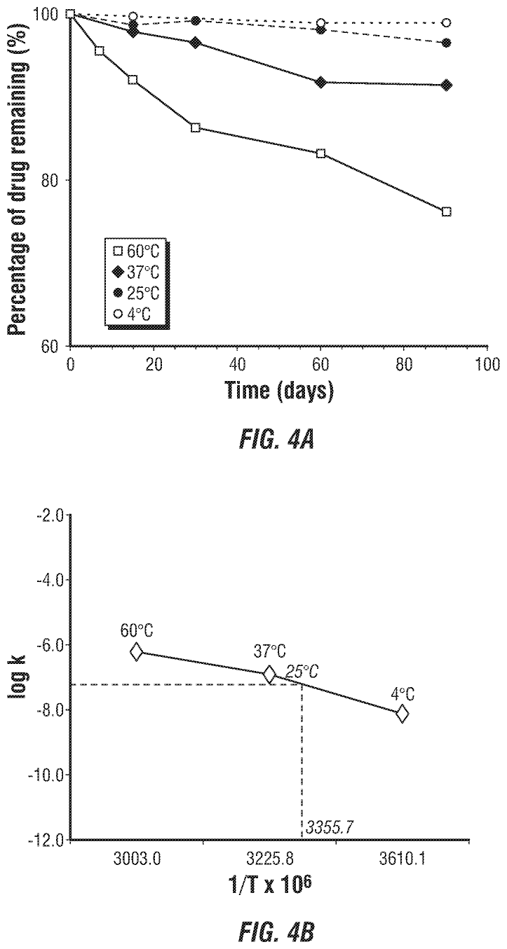

The drug content of the formulation was measured using HPLC immediately after the preparation of the formulation (initial time) and at predetermined time points up to 3 months. The percentage of drug remaining at each time point was plotted against time (FIG. 4A), and the degradation rate constant at each temperature was calculated from the slope of the regression line. A straight slope on logarithmic plot suggested a first order kinetics of degradation in the solution. An Arrhenius plot was generated by plotting the logarithmic first order rate constants against reciprocal of absolute experimental temperatures (FIG. 4B). The rate constants of degradation were derived as 0.0002 day⁻¹ at 25° C. and 0.0001 day⁻¹ at 4° C. from the Arrhenius plot. The time required for riluzole assay to reach 90% of its original value (t₉₀) in the co-solvent formulation were 17 and 35 months at 25° C. (room temperature) and 4° C., respectively. Therefore, the instant co-solvent formulation is stable at room temperature and 4° C.

In Vitro Hemolytic Potential of Co-solvent Formulation: The use of co-solvents is common to improve the solubility of many poorly water soluble compounds. More than 20 known co-solvents are used in parenteral formulations. However, co-solvents have potential risks to cause intravascular hemolysis resulting in unwanted medical complications. Therefore, it is essential to determine the safety of the formulation prior to an intravenous administration. The concentrations of the individual co-solvents in the formulation are below the maximum approved concentrations in intravenous products. However, the safety of the combination of these co-solvents is unknown and needs to be verified. Therefore, the hemolytic potential of this formulation was evaluated before its intravenous administration to the animal model.

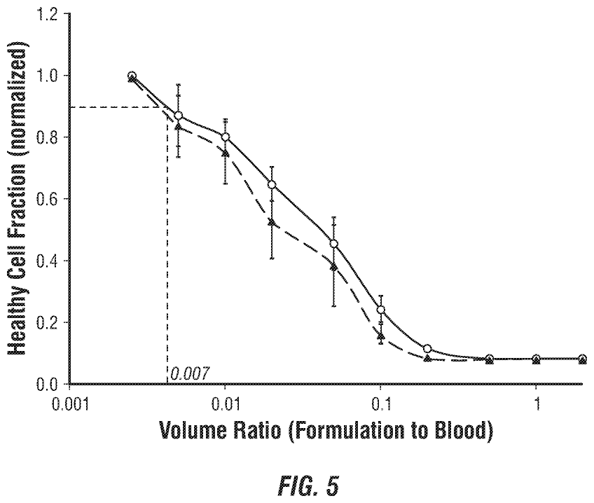

The hemolytic potential of co-solvent formulation was evaluated. The water to blood ratio required to produce complete hemolysis was 10:1, and was further used to prepare standard solutions for generating the standard curve. Hemolytic potential of the co-solvent formulation was evaluated in the formulation: blood (F/B) ratios ranging from 0.0025 to 2.0. The sigmoidal plot (FIG. 5) represents the loss of healthy red blood cell fractions (i.e., increase in hemolysis) with increasing F/B ratios, which is possibly due to the increasing amounts of co-solvents.

The loss of healthy cell fraction was exponentially increased at the F/B ratios of 0.0025 to 0.2, then leveled off when F/B was above 0.2. According to prior studies, formulations exhibiting in vitro hemolysis of <10% should be considered non-hemolytic, and >25% as hemolytic. The optimum co-solvent formulation showed 10-20% hemolysis in the F/B range of 0.005 to 0.01. Based on the blood volume of rats in 250 grams (17.5 ml) and the volume of formulation (125 μl) for the required dose of 5 mg/kg of riluzole, the actual dose to blood ratio is 0.007, at which 90% of cells remain healthy. Therefore, the formulation is non hemolytic at the intended dose level.

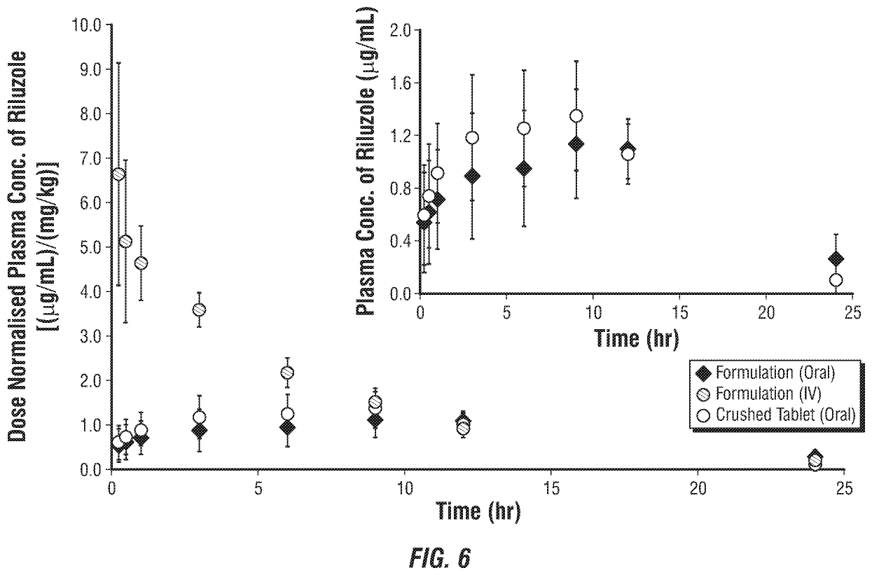

Pharmacokinetic Characterization of Riluzole Formulations: The plasma pharmacokinetics of riluzole was characterized after oral (10 mg/kg) and IV (5 mg/kg) administrations of the co-solvent formulation in rats. The co-solvent formulation was safely and successfully administered intravenously to the rats. The plasma concentration-time profile was constructed up to 24 hours (FIG. 6). The bi-exponential decline in plasma concentration after the IV administration was characterized by an initial short and rapid distribution phase followed by a longer elimination phase. The profile was best described by a two-compartment IV bolus model. The mean distribution half-life was 0.21 hr and mean elimination half-life was 5.42 hours. The PK parameters of riluzole after a single IV dose are tabulated in Table 5. The AUC (mean±SD) was 21.06±1.42 μg*hr/ml.

TABLE 5
Plasma Pharmacokinetic (PK) Parameters of Riluzole from Co-solvent
Formulation (Oral and IV) and Crushed Rilutek ® Tablets.
The PK parameters for orally administered riluzole were derived
using one compartment model with first order absorption and elimination.
The PK parameters for intravenously administered riluzole were
derived using a 2-compartment IV bolus model. Value for each parameter
is represented as Mean ± SD (N = 4-5 in each group).

| | Rilutek ® | | |
|---|---|---|---|
| | Oral | Co-solvent Formulation | |
| PK Parameters | (crushed tablet) | Oral | Intravenous |
| Dose (mg/kg) | 10.0 | 10.0 | 5.0 |
| Absorption rate | 0.90 ± 0.69 | 2.83 ± 1.02* | — |
| constant, K01 | | | |
| (1/hr) | | | |
| Distribution | — | — | 0.21 ± 0.12 |
| half-life (hr) | | | |
| Elimination | 0.10 ± 0.07 | 0.04 ± 0.03 | 0.14 ± 0.02 |
| rate | | | |
| constant, K10 | | | |
| (1/hr) | | | |
| AUC₀₋₂₄ | 27 ± 10 | 39.78 ± 17.89 | 21.06 ± 1.42 |
| (hr*μg/ml) | | | |
| Bioavailability, | 64.10 | 94.44 | — |
| F | | | |
| Absorption | 1.83 ± 1.79 | 0.28 ± 0.12 | — |
| half-life (hr) | | | |
| Elimination | 9.66 ± 5.92 | 25.25 ± 16.09* | 5.42 ± 1.02 |
| half-life (hr) | | | |
| Volume of | 6.33 ± 3.49 | 7.49 ± 2.39 | 1.72 ± 0.40 |
| distribution | | | |
| (L/kg) | | | |
| CL (ml/hr/kg) | 443.99 ± 184.54 | 339.51 ± 249.33 | 238.24 ± 16.50 |

*indicates significant difference at p < 0.05.

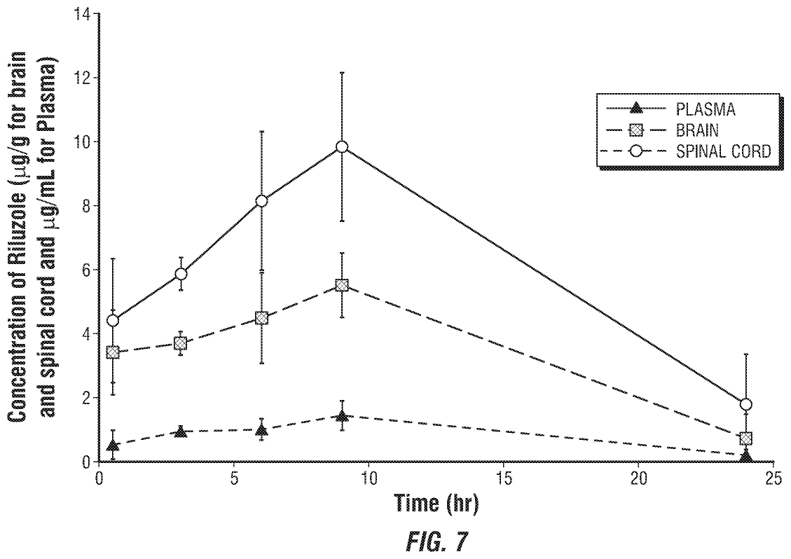

The plasma PK profile of orally administered riluzole (FIG. 6) was best described by one-compartment model with first order absorption (without lag phase) and elimination. The pharmacokinetic parameters derived by this model are given in Table 5. The absorption of riluzole from the co-solvent formulation was rapid and the Cₘₐₓ of 1.33 μg/ml was achieved by 2.2 hours. The concentrations of riluzole were sustained at 1 μg/ml for about 12 hours and then declined. The mean elimination half-life of riluzole from the co-solvent formulation was 5.2 hours, which was almost one-half of the previously reported value of 9-10.2 hours from a cyclodextrin based formulation of riluzole. The average volume of distribution for riluzole form co-solvent formulation was 7.5 L/kg, which indicates extensive distribution of the drug into peripheral tissues. This is desirable since the intended site of action is the central nervous system. A higher concentration of riluzole was observed in brain and spinal cord as compared to plasma at different time points during the study (FIG. 7 and Table 6). This observation is in agreement with previously reported data.

The 24 hour plasma profiles of riluzole were similar between the co-solvent formulation and crushed Rilutek® tablets groups. A point to point comparison of the profiles did not show any statistical significance in plasma concentrations. The PK parameters of both groups are provided in Table 5. The co-solvent formulation group shows significantly faster absorption as compared to the crushed tablet group (absorption half-life: 0.28 vs 1.83 hr). The Cₘₐₓ, Tₘₐₓ and total clearance between the groups were similar. However, the elimination half-life of the formulation group was significantly higher than the crushed tablet (25.5 vs 9.66 hr) group. The AUC of the formulation appeared to be higher as compared to the crushed tablet, but not statistically significant. Thus, the co-solvent formulation provides advantages of faster absorption and sustained levels of riluzole in plasma. This can potentially deliver faster onset and longer duration of action in patients. The absolute bioavailability of the co-solvent formulation and crushed tablet were calculated to be 94.44% and 64.10%, respectively.

As summarized in FIG. 7 and Table 6, the administered co-solvent formulation of riluzole had a significantly higher accumulation in the brain and spinal cords of rats than the administered crushed tablet of Rilutek®.

TABLE 6
Disposition of riluzole in the brain and spinal cord of rats.
B/P and SC/P ratios were measured using plasma, brain and
spinal cord concentrations collected at 24 hr. (n = 5).

| | Rilutek ® | Co-solvent |
|---|---|---|
| Parameters | (crushed tablet) | Formulation |
| Brain to Plasma (B/P) Ratio | 2.33 ± 0.87 | 3.67 ± 1.53 |
| Spinal Cord to Plasma (SC/P) Ratio | 4.09 ± 2.30 | 7.02 ± 2.32 |

### EXAMPLE 1.7

### Summary of Experimental Results

In this Example, Applicants have effectively utilized the central composite design method to optimize the composition of liquid formulation of riluzole containing co-solvents. The aim was to develop a water based formulation for riluzole, using minimum concentrations of co-solvents. An alternative liquid preparation of riluzole is available (Teglutik®, 5 mg/ml) in the form of an oral suspension in some European nations. Although it successfully overcomes the challenges and risks of administering tablets to patients with swallowing dysfunction, it has its own potential limitations as a dosage form. The homogeneity of the suspension has to be ensured visually before administration, which can be biased and result in inconsistent dosing.

In addition, patients have to handle large bulky containers (2×300 ml) of medication since the suspension has a strength of 5 mg/ml. The presently disclosed riluzole solution offers the merit of uniform and homogenous consistency. In addition, the strength is 10 mg/ml, which warrants half-sized containers. The most significant advantage of this formulation is that it can be administered intravenously and hence can be given to critically ill patients.

The selected optimum formulation consists of 15% PEG 400, 20% propylene glycol and 10% glycerin, v/v, with riluzole concentration of 10 mg/ml. It is stable at room temperature and at 4° C. The formulation was well tolerated after both oral and intravenous dosing in rats. Both oral and IV pharmacokinetic profiles for riluzole from the formulation were established. The absolute bioavailability of the oral solution is 94.4%, which is higher than the crushed tablet. When compared to the crushed tablet PK, the developed co-solvent formulation of riluzole shows the merits of yielding a higher bioavailability, faster absorption and a longer elimination half-life, which can potentially result in a higher exposure, a faster onset and a longer duration of action.

In sum, Applicants have successfully developed a stable liquid formulation of riluzole that can be administered both orally and intravenously. It is anticipated that the riluzole liquid formulation could be used in clinical trials in critical care patients such as spinal cord injury patients. In addition, the systematic CCD approach enables Applicants to identify a set of other acceptable liquid formulations (See Table 2) that may be employed for treating various neurological conditions.

More specifically, riluzole was solubilized using water miscible organic solvents, namely, polyethylene glycol 400, propylene glycol and glycerin. The CCD approach was used to develop an optimum co-solvent composition that can solubilize the entire 50 mg dose of riluzole. A three-factor five-level design was employed to investigate the effects of composition of co-solvents on riluzole solubility. The selected optimum formulation consists of 15% v/v PEG 400, 20% v/v propylene glycol and 10% v/v glycerin, with riluzole concentration of 10 mg/ml. The optimum composition was assessed for stability at different temperatures. Satisfactory stability was obtained at room temperature and 4° C. (t90 of 17 and 35 months, respectively). The optimum formulation of riluzole was suitable for both oral and intravenous administrations. Single dose pharmacokinetic studies of the optimum formulation by oral and IV routes were evaluated in rats, using commercially available Rilutek tablets as a reference. The co-solvent formulation was well tolerated both orally and intravenously. In comparison to the commercial tablet, the co-solvent formulation had a faster rate of absorption and more sustained plasma levels with a significantly longer elimination half-life. The riluzole solution formulation is stable and offers advantages of ease of administration, consistent dosing, rapid onset and longer duration of action, which can be extremely beneficial for the therapy in SCI patients.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A composition comprising:
a liquid formulation comprising a molecule,
wherein the molecule is selected from the group consisting of riluzole, a derivative of riluzole, an analog of riluzole, a pharmaceutical equivalent of riluzole, a benzothiazole-based molecule, combinations thereof, and salts thereof.

2. The composition of claim 1, wherein the liquid formulation comprises a liquid selected from the group consisting of solubilizing agents, pharmaceutically acceptable carriers, excipients, syrups, elixir, water, gels, and combination thereof.

3. The composition of claim 1, wherein the liquid formulation has sufficient homogeneity for parenteral administration.

4. The composition of claim 1, wherein the liquid formulation is a colorless solution at room temperature with no visible particles.

5. The composition of claim 1, wherein the molecule has a t₉₀ room temperature stability of more than 10 months.

6. The composition of claim 1, wherein the molecule has a t₉₀ room temperature stability of more than 15 months.

7. The composition of claim 1, wherein the liquid formulation further comprises a solubilizing agent.

8. The composition of claim 7, wherein the solubilizing agent is selected from the group consisting of polyethylene glycol, glycerin, propylene glycol, ethanol, sorbitol, polyoxyethylated glycerides, polyoxyethylated oleic glycerides, polysorbates, sorbitan monooleate, hydroxypropyl-beta-cyclodextrin, polyoxyl 40 hydrogenated castor oil, polyoxyl hydroxystearates, and combinations thereof.

9. The composition of claim 7, wherein the solubilizing agent comprises polyethylene glycol, glycerin, and propylene glycol.

10. The composition of claim 7, wherein the liquid formulation contains less than about 65% v/v of solubilizing agents.

11. The composition of claim 7, wherein the liquid formulation contains less than about 50% v/v of solubilizing agents.

12. The composition of claim 1, wherein the molecule comprises riluzole.

13. The composition of claim 1, wherein the molecule is dissolved in the liquid formulation.

14. The composition of claim 1, wherein the molecule has a concentration of more than 5 mg/ml.

15. The composition of claim 1, wherein the molecule has a concentration of at least about 10 mg/ml.

16. A method of treating a condition or disease in a subject, said method comprising:
administering to the subject a composition,
wherein the composition comprises a liquid formulation comprising a molecule, wherein the molecule is selected from the group consisting of riluzole, a derivative of riluzole, an analog of riluzole, a pharmaceutical equivalent of riluzole, a benzothiazole-based molecule, combinations thereof, and salts thereof.

17. The method of claim 16, wherein the subject is a human

18. The method of claim 16, wherein the condition or disease is selected from the group consisting of spinal cord injury (SCI), swallowing abnormalities, dysphagia, neurological disease or condition, amyotrophic Lateral Sclerosis (ALS), and combinations thereof.

19. The method of claim 16, wherein the condition or disease is spinal cord injury (SCI).

20. The method of claim 16, wherein the condition or disease is dysphagia.

21. The method of claim 16, wherein the administration comprises parenteral administration.

22. The method of claim 21, wherein the parenteral administration is selected from the group consisting of intraorbital administration, infusion, intraarterial administration, intracapsular administration, intracardiac administration, intradermal administration, intramuscular administration, intraperitoneal administration, intrapulmonary administration, intraspinal administration, intrasternal administration, intrathecal administration, intrauterine administration, intravenous administration, subarachnoid administration, subcapsular administration, subcutaneous administration, transmucosal administration, transtracheal administration, intra-articular administration, and combinations thereof.

23. The method of claim 16, wherein the administration comprises oral administration.

24. The method of claim 16, wherein the molecule comprises an absorption half-life of less than 1.5 hours.

25. The method of claim 16, wherein the molecule comprises an absorption half-life of less than 1 hour.

26. The method of claim 16, wherein the molecule comprises an elimination half-life of more than 10 hours.

27. The method of claim 16, wherein the molecule comprises an elimination half-life of more than 15 hours.

28. The method of claim 16, wherein the molecule comprises a bioavailability of more than 65%.

29. The method of claim 16, wherein the molecule comprises a bioavailability of more than 80%.

30. The method of claim 16, wherein the brain (μg/g) to plasma (μg/ml) ratio of the molecule is more than 3.5 after 24 hours of administration.

31. The method of claim 16, wherein the spinal cord (μg/g) to plasma (μg/ml) ratio of the molecule is more than 6.5 after 24 hours of administration.

32. The method of claim 16, wherein the liquid formulation is a colorless solution at room temperature with no visible particles.

33. The method of claim 16, wherein the molecule has a t₉₀ room temperature stability of more than 10 months.

34. The method of claim 16, wherein the molecule has a t₉₀ room temperature stability of more than 15 months.

35. The method of claim 16, wherein the liquid formulation further comprises a solubilizing agent.

36. The method of claim 35, wherein the solubilizing agent is selected from the group consisting of polyethylene glycol, glycerin, propylene glycol, ethanol, sorbitol, polyoxyethylated glycerides, polyoxyethylated oleic glycerides, polysorbates, sorbitan monooleate, hydroxypropyl-beta-cyclodextrin, polyoxyl 40 hydrogenated castor oil, polyoxyl hydroxystearates, and combinations thereof.

37. The method of claim 35, wherein the solubilizing agent comprises polyethylene glycol, glycerin, and propylene glycol.

38. The method of claim 35, wherein the liquid formulation contains less than about 50% v/v of solubilizing agents.

39. The method of claim 16, wherein the molecule comprises riluzole.

40. The method of claim 16, wherein the molecule has a concentration of more than 5 mg/ml.

41. The method of claim 16, wherein the molecule has a concentration of at least about 10 mg/ml.

42. A method of making a composition, wherein the method comprises associating a molecule with a liquid formulation, wherein the molecule is selected from the group consisting of riluzole, a derivative of riluzole, an analog of riluzole, a pharmaceutical equivalent of riluzole, a benzothiazole-based molecule, combinations thereof, and salts thereof.

43. The method of claim 42, wherein the associating occurs by a method selected from the group consisting of mixing, stirring, heating, milling, compressing, and combinations thereof.

44. The method of claim 42, further comprising a step of encapsulating the composition in a carrier.

45. The method of claim 44, wherein the carrier is a capsule.

46. The method of claim 42, wherein the liquid formulation comprises a liquid selected from the group consisting of solubilizing agents, pharmaceutically acceptable carriers, excipients, syrups, elixir, water, gels, and combination thereof.

47. The method of claim 42, wherein the liquid formulation further comprises a solubilizing agent.

48. The method of claim 47, wherein the solubilizing agent is selected from the group consisting of polyethylene glycol, glycerin, propylene glycol, ethanol, sorbitol, polyoxyethylated glycerides, polyoxyethylated oleic glycerides, polysorbates, sorbitan monooleate, hydroxypropyl-beta-cyclodextrin, polyoxyl 40 hydrogenated castor oil, polyoxyl hydroxystearates, and combinations thereof.

49. The method of claim 47, wherein the solubilizing agent comprises polyethylene glycol, glycerin, and propylene glycol.

50. The method of claim 42, wherein the molecule comprises riluzole.

51. The method of claim 42, wherein the molecule becomes dissolved in the liquid formulation.

52. The method of claim 42, wherein the molecule has a concentration of more than 5 mg/ml.
